(19) United States
(12) Patent Application Publication
(10) Pub. No.: US 2002/0082279 A1
(43) Pub. Date: Jun. 27, 2002

(54) METHOD AND COMPOSITION FOR THE TREATMENT OF DERMATOLOGIC DISEASES

(76) Inventor: NEAL B. SCHULTZ, TENAFLY, NJ (US)

Correspondence Address: IRA J SCHAEFER ESQ, CHADBOURNE & PARKE LLP, 30 ROCKEFELLER PLAZA, NEW YORK, NY, 10112

(21) Appl. No.: 09/418,770

(22) Filed: Oct. 15, 1999

(51) Int. Cl. A61K 31/445; A01N 43/40

(52) U.S. Cl. 514/330

(57) ABSTRACT

A topical composition for the treatment of a dermatologic disease includes a carrier, at least one essential oil and a dermatologic active ingredient having a therapeutic effect for the dermatologic disease and a method for treating a dermatologic disease wherein an effective amount of the composition for the disease is applied to a patient having the disease.

### BACKGROUND OF THE INVENTION

The present invention is directed to a method and composition for the treatment of dermatologic diseases.

Alternative medical care is becoming more important and is receiving increasing recognition from established, traditional medical groups in the United States. For example, herbal treatment and specifically the use of essential oils derived from plants have been alleged to have direct therapeutic effects on many organ systems, including the skin, the central nervous system, the cardiovascular system, the respiratory system, the gastrointestinal system, the genito-urinary system, etc.

### SUMMARY OF THE INVENTION

The main object of the present invention is to treat chronic, subacute, and acute conditions and diseases, and in particular, dermatologic conditions and diseases, with medication and essential oils. The combination of the medication and essential oil have a synergy when used to treat dermatologic diseases and conditions. This is accomplished in accordance with the present invention by the topical application of a carrier vehicle, the essential oils and the medication onto the affected area of the skin. The pure essential oil is not applied directly, but dissolved in a carrier vehicle, such as a cream, ointment, lotion, solution, gel, etc., which is then applied to the skin.

In accordance with the invention, the composition can be made by mixing the carrier, oil and medication. Preferably, many premedicated carriers are available to which the essential oils can be added.

The medication is a dermatologic active ingredient that has a recognized therapeutic effect for a skin disease. The active ingredient can be a prescription or nonprescription dermatologic drug.

In addition to direct topical application of an essential oil, an essential oil can also be administered via environmental exposure, for example, incense, potpourri, candles, aerosolizer.

### DETAILED DESCRIPTION OF THE INVENTION

In one embodiment of the present invention, dermatologic disease is treated by combining one or more essential oils which have direct skin effect with a dermatologic active ingredient with recognized therapeutic effect in a carrier vehicle. Preferably, the direct skin effect of the essential oil enhances the dermatologic therapeutic effect of the medication.

In a further embodiment of the present invention, dermatologic disease is treated by combining one or more essential oils which have a central nervous system effect with a dermatologic active ingredient having recognized dermatologic therapeutic effect in a carrier vehicle. The active ingredient can be a prescription or nonprescription drug. Preferably, the central nervous system effect enhances the dermatologic therapeutic effect of the medication.

In another embodiment of the present invention, dermatologic disease is treated by combining an essential oil which has direct skin activity and an essential oil which has central nervous system effects with a dermatologic active ingredient having a recognized dermatologic therapeutic effect in a carrier vehicle. Preferably, at least one of the direct skin effect and the central nervous system effect will enhance the dermatologic therapeutic effect of the active ingredient. The active ingredient can be a prescription or nonprescription drug.

The direct skin effects of the essential oils include antiseptic, anti-anxiety, anti-eczematous, anti-inflammatory, anti-microbial, anti-phlogistic, astringent, bactericidal, bruises, cooling, moisturizing, insect repellant, insecticide, anti-sebhorriac, wound healing promoter, hair improvement (texture, structure and body), anti-infectious, disinfectant, immuno-stimulant, skin/cell rejuvenater, anti-fungal, rubefacient anti-rheumatic and analgesic.

Essential oils with direct skin effects include Bay Leaf, Cinnamon Bark & Leaf, Citronella, Neroli, Peppermint, Juniper Berry, Sandalwood, Chamomile (German & Roman), Cypress, Cajeput, Clove Bud, Elemi, Eucalyptus, Lavender, Lemon Grass, Marjoram (sweet), Myrrh, Rose, Tea Tree, Celery Seed, Geranium Bourbon, Grapefruit, Benzoin Siam, Carrot Seed, Vanilla, Clary Sage, Rosemary, Basil (sweet), Patchouli, Galbanum, Camphor (white), Bergamot, Clary Sage, Lemon, Myrtle, Ylang Ylang, Black Pepper, Rose, Jasmine, Olibanum, Petitgrain, Cedar, Frankincense, Pine, Spruce, Thyme, Wintergreen and Orange.

A test for whether an essential oil has a skin effect comprises using human volunteers who received skin punctures on the forearms. One set of puncture wounds was treated with applications of synthetic essential oils. Natural essential oils were applied on the other wounds. The results showed a faster healing time, including rebuilding of tissue and collagen fibers, on the wounds that were treated with natural essential oils. The central nervous system effects include being psychologically active, effecting emotions, mood and/or psychological parameters and enhancing the therapeutic effect of the medication.

Essential oils with psychological or central nervous system effects are helpful in the treatment of stress management, emotional disorders, such as depression and anxiety, and seasonal affective disorder. Essential oils, by creating an improvement in mood, result in one's thinking and acting more creatively, increasing self-esteem and increasing the likelihood of one being able to help others. All three of these result in positive feedback which creates a greater sense of well being and all contribute to helping with stress management and the other conditions noted hereinbefore. The chronic, subacute and acute dermatologic diseases and conditions being addressed, all have variable etiologic input from stress. A reduction of stress results in an improvement in the dermatologic conditions. In addition, decreasing stress amplifies and synergises with the direct effect of the active prescription ingredient on the dermatologic disease.

The essential oils with psychological activity can be specifically chosen for each individual patient. The two categories of essential oils to consider are those that firstly aromachology research has demonstrated to be generally helpful for these conditions, (e.g., Douglas Fir and Osmanthus) and secondly essentials oils determined by an aromatherapy consultation which are personally helpful to the individual patient predicated on prior conditioning and association, i.e., experiences prior in life. This is determined by taking a history from the patient and testing various essential oils for that patient. The details of the testing process are known to those in the field, however, it should be noted that odor is the most primitive sense and has the deepest ties to old memories which are both positive and negative and therefore relevant to this invention.

Examples of other essential oils which can be used in accordance with the present invention are:

Ajowan, Allspice, Almond Bitter, Ambrette Seed, Amyris, Angelica, Anise Star, Aniseed, Arnica, Asafetida, Balm Lemon, Balsam Canadian, Balsam Copaiba, Balsam Peru, Balsam Tolu, Basil Exotic, Basil French, Bay Laurel, Bay West Indian, Benzoin, Bergamot, Birch Sweet, Birch White, Boldo Leaf, Borneol, Boronia, Broom Spanish, Buchu, Cabreuva, Cade, Cajeput, Calamintha, Calamus, Camphor, Cananga, Caraway, Cardomon, Carrot Seed, Cascarilla, Cassia, Cassie, Cedarwood Atlas, Cedarwood Texas, Cedarwood Virginian, Celery Seed, Chamomile German, Chamomile Maroc, Chamomile Roman, Chervil, Cinnamon, Citronella, Clove, Coriander, Costus, Cubebs, Cumin, Cypress, Deertongue, Dill, Elecampane, Elemi, Eucalyptus Blue Gum, Eucalyptus Lemon-Scented, Eucalyptus Broad-Leaved Peppermint, Fennel, Fir Needle Silver, Frankincense, Galangal, Galbanum, Gardenia, Garlic, Geranium, Ginger, Grapefruit, Guaiacwood, Helichrysum, Hops, Horseradish, Hyacinth, Hyssop, Jaborandi, Jasmine, Juniper, Labdanum, Lavandin, Lavender Spike, Lavender True, Lemon, Lemongrass, Lime, Linaloe, Linden, Litsea Cubeba, Lovage, Mandarin, Marigold, Marjoram Sweet, Mastic, Melilotus, Mimosa, Mint Cornmint, Mint Peppermint, Mint Spearmint, Mugwort, Mustard, Myrrh, Myrtle, Narcissus, Neroli, Niaouli, Nutmeg, Oakmoss, Onion, Opopanax, Orange Bitter, Orange Blossom, Orange Sweet, Oregano Common, Oregano Spanish, Orris, Palmarosa, Parsley, Patchouli, Pennyroyal, Pepper Black, Petitgrain, Pine Dwarf, Pine Longleaf, Pine Scotch, Rose Cabbage, Rose Damask, Rosemary, Rosewood, Rue, Sage Clary, Sage Common, Sage Spanish, Sandalwood, Santolina, Sassafras, Savine, Savory Summer, Savory Winter, Schinus Molle, Snakeroot, Spikenard, Spruce Hemlock, Styrax Levant, Tagetes, Tansy, Tarragon, Tea Tree, Thuja, Thyme Common, Tonka, Tuberose, Turmeric, Turpentine, Valerian, Vanilla, Verbena Lemon, Vetiver, Violet, Wintergreen, Wormseed, Wormwood, Yarrow, Ylang Ylang.

Additional essential oils are St. John's Wort, Spearmint, Rose Otto, Rose Absolute, Ravensave, Pine Black, Pine des Alpes, Pine Sylvestre, Pine Siberian, Amni Visnaga, Basil Holy, Basil Sweet, Basil Tropical, Chamomile Blue, Clary Sage, Cistus (Rock rose), Fir Balsam, Fir Douglas, Fir Grand, Fir Silver, Forest Mint, Hyssop Cineol, Hyssop DeCumbens, Inula, Iris (Orris) CO₂, Eucalyptus Part Unrect., Eucalyptus Citriodora, Eucalyptus Radiata, Eucalyptus Smithii, Lavandin Sweet, Marjoram Spanish, Melissa and Peppermint.

The types of dermatologic diseases and conditions which can be treated in accordance with the present invention include Eczema, Psoriasis, Hives, Rosacea, Impetigo, Acne Vulgaris, Allergic Reactions, Actinic Dermatitis, Alopecia, Atopic Dermatitis, Asteatosis, Balanitis, Burns-1^{st} Degree and 2^{nd} Degree, Candidiasis, Contact Dermatitis, Dyshidrosis, Folliculitis, Insect Bites, Intertrigo, Nummular Eczema, Poison Ivy, Oak and Sumac, Pruritus, Purpura, Scar, Seborrhea, Seborrheic Dermatitis, Sunburn, Tinea Corporis, Tinea Pedis, Tinea Versicolor, Cellulitis, Chemical Burn, Chicken Pox, Herpes Zoster (Shingles), Condyloma Accuminata, Blister, Diaper Dermatitis, Drug Eruption, Erythema Multifare, Excoriation, Furuncle, Hand Eczema, Hematoma, Hemorrhoids, Herpes Simplex, Hyperhidrosis, Hypopigmetation, Hyperpigmentaiton, Keratosis Pilaris, Knuckle Pads, Lichen Planus, Lichen Simplex, Neurodermatitis, Mastitis, Miliaria, Onychomycosis, Parapsoriasis, Perioral Dermatitis, Xerosis, Skin Ulcer, Photodermatitis, Pityriasis Rosea, Pityriasis Alba, Plant Dermatitis, Post Inflammatory Hyperpigmentation, Prurigo Nodularis, Postule, Pyoderma, Radiodermatitis, Rash, Schambergs Disease, Capillaritis, Stasis Dermatitis, Tick Bite, Tinea Cruris, Tinea Manum, Tinea Ungium, Tinea Capitis, Oily Skin, Wrinkles, Dry Hair, Oily Hair, Dandruff, Hair Loss and Chapped Skin.

The prescription and nonprescription medications are those conventionally used for the above-mentioned and other dermatologic diseases.

### EXAMPLES

The topical composition according to the present invention is obtained by mixing a therapeutically effective amount of a dermatologic active ingredient and one or more essential oils with a carrier vehicle comprising a cream, ointment, lotion, solution, gel, etc. The essential oils can be from 0.001 to 20% by weight and preferably about 0.01 to about 3% by weight of the total weight of the topical composition.

Table I lists examples of compositions according to the present invention for the treatment of different diseases. In each of the examples, the carrier vehicle already carries a therapeutically effective amount of the ingredient for the specified disease. The essential oils having a direct skin effect are mixed with the listed medicated carriers to for the composition.

The compositions according to the examples above are prepared by mixing an effective amount of the essential oil into the carrier with the active ingredient until it is totally dissolved. The composition is topically applied to the surface of the skin in the area of the disease and in non-disease areas if appropriate. An effective amount of the composition is applied at prescribed intervals, i.e., once a day or more often if necessary. Where the essential oil enhances the therapeutic effect of the active ingredient, the duration of use to accomplish cure and/or improvement for a treatment of the disease will be shortened from the normal times for that active ingredient.

TABLE I

| | | | Carrier with |
|---|---|---|---|
| | Essential Oil | Carrier with | non- |
| | having direct | prescription | prescription |
| Disease | skin effect | medication | medication |
| Eczema | Lavender (F/M) | Diprolene AF | Acid Mantle |
| | | Cream ™ | Cream ™ |
| Psoriasis | Sandalwood | Psoricon | Estar Gel ™ |
| | (F/M) | Cream ™ | |
| Hives | Chamomile | Lidex Cream ™ | Benadryl |
| | (F/M) | | Cream ™ |
| Rosacea | Geranium | Sulfacet-R | Komed Lotion ™ |
| | Essence (F/M) | Lotion ™ | |
| Impetigo | Tea Tree (F/M) | Bactroban | Aguaphor |
| | | Cream ™ | Healing |
| | | | Ointment ™ |

Table II shows examples of compositions according to the present invention for different diseases, wherein essential oils having central nervous system effect are listed and can be combined with the medicated carriers which are prescription and nonprescription drugs.

TABLE II

| | Essential Oil | | Carrier with |
|---|---|---|---|
| | having central | Carrier with | non- |
| | nervous system | prescription | prescription |
| Disease | effect | medication | medication |
| Eczema | Ylang-Ylang | Diprolene AF | Lubriderm |
| | (F/M) | Cream ™ | Lotion ™ |
| Psoriasis | Douglas Fir | Psoricon | Estar Gel ™ |
| | (M) | Cream ™ | |
| | Rose Oil (F) | | |
| Hives | Neroli Oil | Lidex Cream ™ | Benadryl |
| | (F/M) | | Cream ™ |

Table III shows examples of compositions according to the present invention, wherein diseases are treated by the use of both essential oils having direct skin effect and essential oils having central nervous system effect which can be combined with the medicated carriers which are prescription or nonprescription drugs.

TABLE III

| | Essential Oils | | |
|---|---|---|---|
| | having direct | | |
| | skin effect | | Carrier with |
| | and central | Carrier with | non- |
| | nervous system | prescription | prescription |
| Disease | effect | medication | medication |
| Eczema | Lavender (F/M) | Diprolene AF | Lubriderm |
| | Ylang-Ylang | Cream ™ | Lotion ™ or |
| | (F/M) | | Acid Mantle |
| | | | Cream ™ |
| Psoriasis | Sandalwood | Psoricon | Estar ™ |
| | (F/M) | Cream ™ | Gel ™ |
| | Douglas Fir | | |
| | (M) | | |
| | Rose Oil (F) | | |
| Hives | Chamomile | Lidex Cream ™ | Benadryl |
| | (F/M) | | Cream ™ |
| | Neroli Oil | | |
| | (F/M) | | |

The essential oils listed in Tables I-III indicate whether the essential oil is preferable for male, female or both male and female.

What is claimed is:

1. A topical composition for the treatment of a dermatologic disease, comprising: a carrier; at least one essential oil; and a therapeutically effective amount of a dermatologic active ingredient having a therapeutic effect for the dermatologic disease.

2. The composition according to claim 1, wherein the active ingredient is a prescription drug.

3. The composition according to claim 1, wherein the active ingredient is a non-prescription drug.

4. The composition according to claim 2 or 3, wherein the carrier is a cream, ointment, lotion, gel or solution.

5. The composition according to claim 2 or 3, wherein the at least one essential oil has a direct skin effect.

6. The composition according to claim 2 or 3, wherein the at least one essential oil has a central nervous system effect.

7. The composition according to claim 2 or 3, wherein the at least one essential oil has a direct skin effect and has a central nervous system effect.

8. The composition according to claim 5, wherein the direct skin effect enhances the therapeutic effect of the active ingredient.

9. The composition according to claim 6, wherein the central nervous system effect enhances the therapeutic effect of the active ingredient.

10. The composition according to claim 7, wherein at least one of direct skin effect and the central nervous system effect enhances the therapeutic effect of the active ingredient.

11. The composition according to claim 1, for the treatment of eczema, psoriasis, hives, rosacea or impetigo.

12. A method for treating a dermatologic disease, comprising topically applying an effective amount therefor of the composition according to claim 1 to a patient having said dermatologic disease.

13. The method according to claim 12, wherein the active ingredient is a prescription drug.

14. The method according to claim 12, wherein the active ingredient is a non-prescription drug.

15. The method according to claim 13 or 14, wherein the carrier is a cream, ointment, lotion, gel or solution.

16. The method according to claim 13 or 14, wherein the at least one essential oil has a direct skin effect.

17. The method according to claim 13 or 14, wherein the at least one essential oil has a central nervous system effect.

18. The method according to claim 13 or 14, wherein the at least one essential oil has a direct skin effect and has a central nervous system effect.

19. The method according to claim 16, wherein the direct skin effect enhances the therapeutic effect of the active ingredient.

20. The method according to claim 17, wherein the central nervous system effect enhances the therapeutic effect of the active ingredient.

21. The method according to claim 18, wherein at least one of direct skin effect and the central nervous system effect enhances the therapeutic effect of the active ingredient.

22. The method according to claim 12, for the treatment of eczema, psoriasis, hives, rosacea or impetigo.
